(19) United States
(12) Patent Application Publication
(10) Pub. No.: US 2024/0279604 A1
(43) Pub. Date: Aug. 22, 2024

(54) 3D spheroid having uniform size and composition comprising neuron and astrocyte derived from Alzheimer's disease patients, method for producing thereof, and method and kit for screening drug using thereof

(71) Applicant: Industry-University Cooperation Foundation Hanyang University Erica Campus, Ansan-si (KR)

(72) Inventors: Chong Suk RYOU, Yongin-si (KR); Hyun Jung PARK, Anyang-si (KR)

(21) Appl. No.: 18/582,703

(22) Filed: Feb. 21, 2024

(30) Foreign Application Priority Data
Feb. 21, 2023 (KR) 10-2023-0023005

(51) Int. Cl. C12N 5/0793; C12M 1/00; C12N 5/00; C12N 5/079

(52) U.S. Cl. CPC C12N 5/0619; C12M 23/02; C12N 5/0018; C12N 5/0622; C12N 2500/32; C12N 2500/34; C12N 2501/115; C12N 2503/02; C12N 2506/08; C12N 2513/00

(57) ABSTRACT

The present invention relates to a method for producing a spheroid including neurons and astrocytes, the method including: culturing induced pluripotent stem cell-neural progenitor cells (iPSC-NPCs) in an NPC medium; culturing the cultured cells in a neuronal differentiation medium (NDM); and culturing the cultured cells in a glial differentiation medium (GDM). The spheroid produced by the method according to the present invention contains mature neurons and astrocytes which express a desired marker after 3 weeks of in vitro culture and has a uniform size, and thus can be used for simple visual screening to confirm drug efficacy.

### CROSS-REFERENCE TO RELATED APPLICATION

This application claims priority to and the benefit of Korean Patent Application No. 2003-0023005, filed on Feb. 21, 2023, the disclosure of which is incorporated herein by reference in its entirety.

### BACKGROUND

### Field of the Invention

The present invention relates to a 3D spheroid of neurons and astrocytes with a uniform size and composition derived from Alzheimer's disease patients, a method for producing the same, and a method and a kit for screening a drug using the same.

### Discussion of Related Art

Alzheimer's disease is the most common cause of age-related dementia, and its incidence rate is increasing as the population ages. Alzheimer's disease is characterized by a decline in cognitive ability, leading to progressive memory loss and a consequent loss of functional independence. Therefore, there is a need for drugs that slow or stop the progression of Alzheimer's disease.

In order to develop an effective drug, a disease model that exhibits the key features of Alzheimer's disease is essential. A number of genetically engineered mouse models including mutations associated with familial Alzheimer's disease (FAD) have been presented, and these models exhibit some of the pathological and behavioral signs of human Alzheimer's disease. For example, genetically engineered mice with mutations in the amyloid beta (Aβ) precursor protein (APP) and/or presenilin (PS) exhibit three major pathological hallmarks of Alzheimer's disease brains, that is, gradual accumulation of extracellular Aβ, intracellular hyperphosphorylated tau (pTau) (neurofibrillary tangles; NFT), and associated neuroinflammation. However, the Alzheimer's disease-like neuropathological characteristics observed in these models differ from Alzheimer's disease in humans in many ways, such as neuroinflammatory responses and gene expression profiles. Therefore, there is a problem in that the efficacy of Alzheimer's disease drugs may appear differently in mice and humans.

As an alternative to animal models, many research groups have developed 3D organoids derived from patient progenitor cells, such as induced pluripotent stem cells (iPSCs) which express FAD- or sporadic Alzheimer's disease (SAD)-related genes (Jorfi M, D'Avanzo C, Tanzi R E, Kim D Y, Irimia D. Human neurospheroid arrays for in vitro studies of Alzheimer's disease. Sci Rep. 2018; 8(1):2450; Kim Y H, Choi S H, D'Avanzo C, et al. A 3D human neural cell culture system for modeling Alzheimer's disease. Nat Protoc. 2015; 10(7):985-1006; Lee H K, Velazquez Sanchez C, Chen M, et al. Three dimensional human neuro-spheroid model of Alzheimer's disease based on differentiated induced pluripotent stem cells. PLOS One. 2016; 11(9): e0163072; Park J, Wetzel I, Marriott I, et al. A 3D human triculture system modeling neurodegeneration and neuroinflammation in Alzheimer's disease. Nat Neurosci. 2018; 21(7):941-951; Shin Y, Choi S H, Kim E, et al. Blood-brain barrier dysfunction in a 3D in vitro model of Alzheimer's disease. Adv Sci. 2019; 6(20):1900962). These organoids include cultured spheroids, 3D bioprinted brain tissue, brains-on-a-chip, and various combined platforms and exhibit key features of Alzheimer's disease, including Aβ-plaque deposition, NFT formation, and neurodegenerative inflammation. Human iPSC-derived 3D organoids retain intact human genetic characteristics including pathological variations and thus are a particularly powerful model for pathological studies, enable large-scale drug screening, can be scaled up in vitro, and enable multiple experimental repetitions and measurement techniques (Israel M A, Yuan S H, Bardy C, et al. Probing sporadic and familial Alzheimer's disease using induced pluripotent stem cells. Nature. 2012; 482(7384):216-220; Kondo T, Asai M, Tsukita K, et al. Modeling Alzheimer's disease with iPSCs reveals stress phenotypes associated with intracellular Abeta and differential drug responsiveness. Cell Stem Cell. 2013; 12(4):487-496; Muratore C R, Rice H C, Srikanth P, et al. The familial Alzheimer's disease APPV717I mutation alters APP processing and Tau expression in iPSC-derived neurons. Hum Mol Genet. 2014; 23(13):3523-3536; Sproul A A, Jacob S, Pre D, et al. Characterization and molecular profiling of PSEN1 familial Alzheimer's disease iPSC-derived neural progenitors. PLOS One. 2014; 9(1): e84547; Yagi T, Ito D, Okada Y, et al. Modeling familial Alzheimer's disease with induced pluripotent stem cells. Hum Mol Genet. 2011; 20(23): 4530-4539). However, for accurate contrast between experiments, it should be possible to prepare a consistent cell composition while preparing cell-based models of sufficient size, and the fundamental cellular structure and gene expression profile of the human brain structure needs to be maintained, which is recognized as a technically very difficult problem.

### RELATED ART DOCUMENTS

### Non-Patent Documents

Israel M A, Yuan S H, Bardy C, et al. Probing sporadic and familial Alzheimer's disease using induced pluripotent stem cells. Nature. 2012; 482(7384):216-220
Kondo T, Asai M, Tsukita K, et al. Modeling Alzheimer's disease with iPSCs reveals stress phenotypes associated with intracellular Abeta and differential drug responsiveness. Cell Stem Cell. 2013; 12(4):487-496
Muratore C R, Rice H C, Srikanth P, et al. The familial Alzheimer's disease APPV717I mutation alters APP processing and Tau expression in iPSC-derived neurons. Hum Mol Genet. 2014; 23(13):3523-3536
Sproul A A, Jacob S, Pre D, et al. Characterization and molecular profiling of PSEN1 familial Alzheimer's disease iPSC-derived neural progenitors. PLOS One. 2014; 9(1): e84547
Yagi T, Ito D, Okada Y, et al. Modeling familial Alzheimer's disease with induced pluripotent stem cells. Hum Mol Genet. 2011; 20(23): 4530-4539

### SUMMARY OF THE INVENTION

The present invention is directed to providing a 3D spheroid derived from human iPSC-NPCs, which satisfies the morphological, functional, and structural characteristics required by drug development platforms.

The present invention is also directed to providing a method for producing a spheroid including neurons and astrocytes, the method including: culturing induced pluripotent stem cell-neural progenitor cells (iPSC-NPCs) in a neural progenitor cell (NPC) medium; culturing the cultured cells in a neuronal differentiation medium (NDM); and culturing the cultured cells in a glial differentiation medium (GDM).

In an embodiment of the present invention, the iPSC-NPCs are derived from a healthy person or Alzheimer's disease patient. In an embodiment of the present invention, the Alzheimer's disease patient has a presenilin1 (PS1) gene mutation. In an embodiment of the present invention, the NPC medium includes non-essential amino acids, sodium pyruvate, glucose, glutamine, beta-methanol, and bFGF. In an embodiment of the present invention, the culturing in the NPC medium is performed in a U-shaped bottom plate for 2 to 4 days. In an embodiment of the present invention, the culturing in the NDM is performed in a U-shaped bottom plate for 6 to 8 days. In an embodiment of the present invention, the culturing in the GDM is performed in a U-shaped bottom plate for 12 to 16 days.

According to an aspect of the present invention, there is provided a spheroid including neurons and astrocytes, which is produced by a method including: culturing induced pluripotent stem cell-neural progenitor cells (iPSC-NPCs) in an NPC medium; culturing the cultured cells in a neuronal differentiation medium (NDM); and culturing the cultured cells in a glial differentiation medium (GDM). In an embodiment of the present invention, the iPSC-NPCs are derived from a healthy person or Alzheimer's disease patient. In an embodiment of the present invention, the Alzheimer's disease patient has a presenilin1 (PS1) gene mutation. In an embodiment of the present invention, the NPC medium includes non-essential amino acids, sodium pyruvate, glucose, glutamine, beta-methanol, and bFGF. In an embodiment of the present invention, the culturing in the NPC medium is performed in a U-shaped bottom plate for 2 to 4 days. In an embodiment of the present invention, the culturing in the NDM is performed in a U-shaped bottom plate for 6 to 8 days. In an embodiment of the present invention, the culturing in the GDM is performed in a U-shaped bottom plate for 12 to 16 days. In an embodiment of the present invention, the spheroid includes mature neurons and mature astrocytes at a ratio of 1.2:1 to 1.6:1.

According to another aspect of the present invention, there is provided a method for screening a therapeutic substance for Alzheimer's disease, the method including: treating a spheroid including neurons and astrocytes with a candidate substance; and confirming at least one selected from the group consisting of Aβ plaque deposition, formation of hyperphosphorylated tau, and neurodegenerative inflammation, wherein the spheroid is produced by a method including: culturing induced pluripotent stem cell-neural progenitor cells (iPSC-NPCs) derived from an Alzheimer's disease patient in an NPC medium; culturing the cultured cells in a neuronal differentiation medium (NDM); and culturing the cultured cells in a glial differentiation medium (GDM). In an embodiment of the present invention, when Aβ plaque deposition, formation of hyperphosphorylated tau, or neurodegenerative inflammation is reduced in a group treated with the candidate substance compared to a group not treated with the candidate substance, it is determined that the candidate substance is a therapeutic substance for Alzheimer's disease. In an embodiment of the present invention, the spheroid includes mature neurons and mature astrocytes at a ratio of 1.2:1 to 1.6:1.

According to another aspect of the present invention, there is provided a kit for screening a therapeutic substance for Alzheimer's disease, including a spheroid including neurons and astrocytes, which is produced by a method including: culturing induced pluripotent stem cell-neural progenitor cells (iPSC-NPCs) derived from an Alzheimer's disease patient in an NPC medium; culturing the cultured cells in a neuronal differentiation medium (NDM); and culturing the cultured cells in a glial differentiation medium (GDM). In an embodiment of the present invention, the spheroid includes mature neurons and mature astrocytes at a ratio of 1.2:1 to 1.6:1.

### Effect of the Invention

The spheroid according to the present invention contains mature neurons and astrocytes expressing desired markers after 3 weeks of in vitro culture and has a uniform size, and therefore, it can be used for simple visual screening to confirm drug efficacy.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 relates to the selection of a culture solution for co-culturing a 3D spheroid of neurons and astrocytes. (A) relates to a schematic method for producing an iPSC-NPC-derived spheroid. iPSC-NPCs derived from a normal group were cultured in an NPC medium for 3 days, and aggregated cells were cultured under either condition for 3 weeks. Aggregated cells of Group 1 were cultured in a neuronal medium for 21 days, and aggregated cells of Group 2 were cultured in a neuronal medium for 7 days and in an astrocyte medium for 14 days. (B) shows the results of confirming the cell composition constituting the spheroid by the expression of molecular markers (positive markers: NESTIN, MUSASHI, and SOX2; negative marker: MAP2). (C) is a set of photographs taken of Group 1 and Group 2 spheroids in vitro after 24 days. There were no significant differences between Group 1 and Group 2 in the area and perimeter of the spheroid (n=5). (D) shows the confirmation of neuronal differentiation of Group 1 and Group 2 spheroids through immunostaining for a neuron marker (MAP2) and an astrocyte marker (GFAP). It was found that the spheroid of Group 2 was uniformly surrounded by astrocytes, and neurons had a large number of MAP2-positive dendrites. (E) shows the results of confirming the expression levels of mature neuron markers MAP2 and synaptophysin and mature astrocyte markers GFAP and Kir4.1 in the spheroids of Group 1 and Group 2 by western blotting (*p<0.05, **p<0.01, and ***p<0.001). All indicators (scale bars) in FIG. 1 are 100 μm.

FIG. 2 shows the experimental results for confirming the optimal number of cells at the start of culture for producing 3D spheroids. (A) Cell aggregates formed from 3000, 5000, 30000, and 50000 iPSC-NPCs derived from a normal individual were photographed on Days 3, 10, and 24 after culture. The indicator is 100 μm. (B) shows the average spheroid area. Through this, 50,000 cells were determined as the number of starting cells for producing a spheroid (n=3).

FIG. 3 shows the results of quantifying the cell composition of spheroids through RT-PCR. (A) shows the results of seeding iPSC-NPCs derived from a normal donor (control; CTL) into a 96-well U-shaped bottom plate and measuring the spheroid area and perimeter after 3 weeks of culture. The area and perimeter of the spheroid were shown to be relatively constant (n=29). (B) shows the results of performing a single-cell phenotypic analysis on each spheroid through marker expression profiles. As a result of confirmation by a marker expression heatmap, 18,401 cells were composed of 3% immature astrocytes, 9% immature neurons, 46% mature neurons, and 32% mature astrocytes. (C) shows the results of RT-PCR analysis of TUBB1, MAP2, NEFM, NEFH, GFAP, S100, and GLUL expression in three spheroids. No significant differences in expression levels were found, indicating a relatively uniform cell phenotypic composition.

FIG. 4 shows the results of Aβ accumulation, cell death by ThT, and activated caspase staining, respectively. (A) shows the results of detecting Aβ aggregates in a control 3D spheroid treated with aggregated Aβ through ThT staining. (B) shows the results of detecting Aβ aggregates in a control 3D spheroid treated with aggregated Aβ through Aβ antibody (6E10) staining. (C) shows the results of confirming cell viability and apoptosis in a control spheroid using an activated caspase assay. (D) shows the results of quantifying total caspase 3 and cleaved caspase 3 in Aβ-treated 3D spheroids through western blotting. Expression levels showed decreased levels of total caspase 3 and increased levels of cleaved caspase 3 compared to 3D spheroids without Aβ treatment. (*p<0.05, ***p<0.001, and ****p<0.0001). All indicators (scale bars) in FIG. 4 are 100 μm.

FIG. 5 shows the results of confirming the morphology, differentiation state, and pathological characteristics of 3D spheroids derived from Alzheimer's disease patients. (A) shows the results of producing 3D spheroids from iPSC-NPCs of Alzheimer's disease patients using the same method as that used for control spheroids. Similar to the control, spheroids derived from Alzheimer's disease patients showed a relatively uniform size (n=36). (B) shows the results of confirming the differentiation state of 3D spheroids derived from Alzheimer's disease patients through MAP2 and GFAP immunofluorescence. MAP2-positive dendrites of neurons were shorter and GFAP expression was stronger than those of control 3D spheroids (CTL: control group; N1 and N2: randomly selected independent spheroids). (C) shows that spheroids derived from Alzheimer's disease patients exhibit stronger basal immunoexpression of Aβ (stained with Aβ6E10 antibody) and the expression is distributed throughout the spheroids. Aβ (1-42) was quantified by ELISA. The expression level of Aβ (1-42) was shown to be higher than that in the control 3D spheroids. (D) shows the results of quantifying astrocyte marker GFAP and reactive astrocyte markers C3 and IL-6 through western blotting. The expression levels of GFAP, C3 and IL-6 were shown to be higher in spheroids derived from Alzheimer's disease patients than in control 3D spheroids (**p<0.01). All indicators (scale bars) in FIG. 5 are 100 μm.

FIG. 6 shows the ThT and activated caspase staining results of 3D spheroids derived from Alzheimer's disease patients to evaluate the efficacy of anti-Alzheimer's drugs. (A) shows the results of a ThT fluorescence assay on Aβ (1-42) aggregates. It could be confirmed that the formation of Aβ (1-42) aggregates was suppressed by administering 10 μM NDGA or CU. (B) shows the results of quantifying Aβ (1-42) by ELISA. As a result of culturing Alzheimer's disease-derived spheroids with NDGA and CU, the level of Aβ (1-42) aggregates was reduced. (C) shows the results of confirming changes in Aβ aggregates using ThT staining of Alzheimer's disease-derived spheroids cultured with NDGA and CU. When treated with NDGA and CU, ThT staining intensity was reduced in 3D Alzheimer's disease-derived spheroids. (D) shows the results of confirming changes in cell death using an activated caspase assay in Alzheimer's disease-derived 3D spheroids cultured with NDGA or CU. Activated caspase cell death staining was reduced in all groups treated with NDGA and CU (*p<0.05, **p<0.01, and ****p<0.0001). All indicators (scale bars) in FIG. 6 are 100 μm. (CTL: control; N1, N2 and N3: randomly selected independent spheroids)

FIG. 7 shows the confocal z-stack intensity of activated caspase in Alzheimer's disease-derived 3D spheroids cultured with NDGA or CU. Cell penetration/uptake ability was confirmed using confocal z-stack (μm) images through CellEvent staining. (A) shows Alzheimer's disease-derived spheroids, (B) shows Alzheimer's disease-derived spheroids treated with NDGA, and (C) Alzheimer's disease-derived spheroids treated with CU.

### DETAILED DESCRIPTION OF EXEMPLARY EMBODIMENTS

Hereinafter, the present invention will be described in detail through embodiments of the present invention with reference to the accompanying drawings. However, the following embodiments are presented as examples of the present invention, and when it is determined that a detailed description of well-known technologies or configurations known to those skilled in the art may unnecessarily obscure the gist of the present invention, the detailed description thereof may be omitted, and the present invention is not limited thereto. Various modifications and applications of the present invention are possible within the description of claims to be described below and the equivalent scope interpreted therefrom.

Further, the terminology used herein includes terms used to properly express preferred embodiments of the present invention, which may vary according to a user's or operator's intention, or customs in the art to which the present invention pertains. Accordingly, definitions of the terms need to be described based on contents throughout the present specification. Throughout the specification, when one part “includes” a certain element, unless specifically stated to the contrary, this does not mean that another constituent element is excluded, but means that another constituent element may be further included.

All technical terms used in the present invention are used in the same sense as those generally understood by one skilled in the art related to the present invention, unless otherwise defined. Further, in the present specification, a preferred method or sample is described, but those similar or equivalent thereto also fall within the scope of the present invention. The contents of all the publications described as a reference document in the present specification are incorporated into the present invention by reference.

As used herein, the term “about” is used to refer to a value such as size and time, and includes an error range of 20%, +10%, +5%, ±1%, or ±0.1% of a specific value. Those skilled in the art can expect to be able to achieve the objectives of the present invention within the scope.

As used herein, the terms “measuring” or “detecting” or terms of similar meaning mean confirming the presence, absence, or amount of a substance or phenomenon of interest.

The present invention relates to a method for producing a spheroid including neurons and astrocytes, the method including: culturing induced pluripotent stem cell-neural progenitor cells (iPSC-NPCs) in a neural progenitor cell (NPC) medium; culturing the cultured cells in a neuronal differentiation medium (NDM); and culturing the cultured cells in a glial differentiation medium (GDM).

As used herein, the term “induced pluripotent stem cells” or “iPSCs” is known in the art and refers to cells having properties similar to embryonic stem cells (ESCs), and encompasses undifferentiated cells artificially produced by reprogramming differentiated, non-pluripotent cells, especially adult somatic cells.

As used herein, the term “pluripotent stem cells” (PSCs) refers to cells capable of continuous self-renewal and generating progeny cells of several different cell types under suitable conditions. Pluripotent stem cells are capable of generating progeny cells that are three germ layer derivatives of the endoderm, mesoderm, and ectoderm. Pluripotent stem cells include various types of embryonic cells such as embryonic stem cells (ESCs) and induced pluripotent stem cells (iPSCs).

Those skilled in the art will understand that PSCs include derivatives such as established cell lines with early tissue and phenotypic characteristics of PSCs and cell lines capable of still generating progeny cells of each of the three germ layers. In a PSC culture solution, a substantial portion of stem cells and derivatives thereof, which exhibit morphological characteristics of undifferentiated cells that are clearly distinguishable from differentiated cells of embryonic or adult origin, is expressed as “undifferentiated” or “substantially undifferentiated.” Undifferentiated PSCs will be easily recognized by those skilled in the art.

The iPSCs used in the present invention may be derived from a healthy person or Alzheimer's disease patient, and may be obtained from, for example, fibroblasts, adipocytes, or the like. The Alzheimer's disease patient may include mutations in one or more genes selected from the group consisting of APP, FNBPIL, SELIL, LINC00298, PRKCH, C15ORF41, C2CD3, KIF2A, APC, LHX9, NALCN, CTNNA2, SYTL3, CLSTN2, PSEN1 (or PS1), and PSEN2 (or PS2) (Prokopenko D, Morgan S L, Mullin K, et al. Whole-genome sequencing reveals new Alzheimer's disease-associated rare variants in loci related to synaptic function and neuronal development. Alzheimer's Dement. 2021; 1-19).

As used herein, the term “neural progenitor cells” or “NPCs” refers to a cell type capable of generating cells of the nervous system including neurons and glial cells, such as, for example, astrocyte progenitor cells, astrocytes, oligodendrocyte progenitor cells, and oligodendrocytes, through differentiation, but is not limited thereto. Progenitor cells are stem cell-like cells, but have lower replication and proliferation capabilities than stem cells. However, compared to fully differentiated cells, progenitor cells still have the ability to differentiate into various cell types. As used herein, the term “iPSC-NPCs” refers to neural progenitor cells derived from induced pluripotent stem cells.

A method for producing iPSC-NPCs may be performed according to methods known in the art. For example, iPSC-NPCs are produced by culturing undifferentiated cells in mTeSR Plus (STEMCELL Technologies, 05825) on a Matrigel-coated plate, seeding the cultured undifferentiated cells into an NPC differentiation medium at 16,000 cells/cm², and then replacing the medium with a fresh medium every day while culturing the resulting cells, and collecting the cultured cells after about 8 days. As the NPC differentiation medium used here, DEM/F12 (Thermo Fisher, 11320033) containing 5 μg/ml insulin, 64 g/ml L-ascorbic acid, 14 ng/ml sodium selenite, 10.7 μg/ml holo-transferrin, 543 μg/ml sodium bicarbonate, 10 μM SB431542 and 100 ng/ml noggin may be used, but is not limited thereto.

In an embodiment of the present invention, a spheroid in which neurons and astrocytes are spatially arranged in a constant manner at a predetermined ratio may be produced by sequentially culturing and differentiating iPSC-NPC cells in an NPC medium, a neuronal differentiation medium (NDM), and a glial differentiation medium (GDM).

In an embodiment of the present invention, the NPC medium may be one that is known in the art and commercially available. For example, the NPC medium may include non-essential amino acids, sodium pyruvate, glucose, glutamine, beta-methanol, and bFGF. In an embodiment of the present invention, the NPC medium may include 1:100 antifungal-antibiotic (Welgene, Korea), 1:100 non-essential amino acids, sodium pyruvate (GIBCO, Waltham, MA), D-glucose (Sigma-Aldrich, Burlington, MA), L-glutamine (Welgene), 1:1000 beta-methanol (Sigma-Aldrich), 1:50 B-27 (without vitamin A; GIBCO), and DMEM/F12 supplemented with 20 ng/ml bFGF, or may include DMEM/F12, N2 and B27, 20 ng/ml FGF, and 500 ng/ml EGF, but is not limited thereto.

In an embodiment of the present invention, the neuronal differentiation medium, that is, NDM, may be one known in the art. For example, the NDM may be DMEM F12 and/or a Neurobasal-based medium including L-glutamine, vitamins, and proteins. In an embodiment of the present invention, the medium may further include a supplement, and the supplement includes corticosterone, D-galactose, ethanolamine, HCl, glutathione, L-carnitine HCl, linoleic acid, linolenic acid, progesterone, putrescine 2HCl, sodium selenite, and/or triiodothyronine (T3). In an embodiment of the present invention, the vitamin may include biotin, DL alpha tocopherol acetate, DL alpha tocopherol, and/or vitamin A. In an embodiment of the present invention, the protein included in the NDM may include BSA, catalase, human recombinant insulin, human transferrin, and/or superoxide dismutase. In an embodiment of the present invention, as the supplement, B27 supplement (Gibco) was used. In an embodiment of the present invention, NDM may include Glutamax™, DMEM F12 supplemented with 1% B27 supplement containing no vitamin A, and Neurobasal™. The Glutamax provides L-alanyl-L-glutamine dipeptide instead of L-glutamine. The compositions of DMEM F12 and Neurobasal may be confirmed by the manufacturer.

In an embodiment of the present invention, the glial differentiation medium, that is, GDM, may be bovine fetal serum and a commercially available astrocyte medium. In an embodiment of the present invention, the GDM may further include an antifungal and/or an antibiotic to prevent contamination, and may further include a supplement. In an embodiment of the present invention, the supplement may be Astrocyte Growth Supplement (Cat #1852) manufactured by ScienCell Research Laboratories, but is not limited thereto. In an embodiment of the present invention, the astrocyte medium may be Astrocyte Medium (Cat #1801) sold by ScienCell Research Laboratories, but is not limited thereto. Information on each product may be provided by the manufacturer.

The non-essential amino acids include one or more amino acids selected from the group consisting of alanine, arginine, aspartic acid, cysteine, glutamic acid, glutamine, glycine, proline, serine, and tyrosine.

In an embodiment of the present invention, a round bottom-shaped plate may be used to culture cells into spheroids, and in particular, in the present invention, a U-shaped bottom plate was used at each culture stage.

In an embodiment of the present invention, cells are cultured in the NPC medium for 2 days to 4 days, 2 days to 3 days, 3 days to 4 days, or about 3 days, in the NDM for 6 days to 8 days, 6 days to 7 days, 7 days to 8 days, or 7 days, in the GDM for 12 days to 16 days, 12 days to 15 days, 12 days to 14 days, 13 days to 16 days, 13 days to 15 days, 13 days to 14 days, or about 14 days.

An embodiment of the present invention provides a spheroid including neurons and astrocytes, which is produced by a method including: culturing induced pluripotent stem cell-neural progenitor cells (iPSC-NPCs) in an NPC medium; culturing the cultured cells in a neuronal differentiation medium (NDM); and culturing the cultured cells in a glial differentiation medium (GDM). The spheroid includes mature neurons and mature astrocytes at a ratio of 1.2:1 to 1.6:1, 1.2:1 to 1.5:1, 1.2:1 to 1.45:1, 1.3:1 to 1.6:1, 1.3:1 to 1.5:1, 1.3:1 to 1.45:1, or 1.4:1 to 1.45:1. In an embodiment of the present invention, based on the number of cells, mature neurons account for about 46% of the spheroid, and mature astrocytes account for about 32% of the spheroid.

An embodiment of the present invention provides a method for screening a therapeutic substance for Alzheimer's disease, the method including: treating a spheroid including neurons and astrocytes with a candidate substance; and confirming at least one selected from the group consisting of Aβ plaque deposition, formation of hyperphosphorylated tau, and neurodegenerative inflammation, wherein the spheroid is produced by a method including: culturing induced pluripotent stem cell-neural progenitor cells (iPSC-NPCs) derived from Alzheimer's disease patients in an NPC medium; culturing the cultured cells in a neuronal differentiation medium (NDM); and culturing the cultured cells in a glial differentiation medium (GDM).

For example, among the spheroids, when Aβ plaque deposition, formation of hyperphosphorylated tau, or neurodegenerative inflammation is reduced in a group treated with the candidate substance compared to a group not treated with the candidate substance, the candidate substance may be determined to be a therapeutic substance for Alzheimer's disease. Alternatively, for example, among the spheroids, when the expression of modified APP, FNBPIL, SELIL, LINC00298, PRKCH, C15ORF41, C2CD3, KIF2A, APC, LHX9, NALCN, CTNNA2, SYTL3, CLSTN2, PSEN1 (or PS1), or PSEN2 (or PS2) is reduced in a group treated with the candidate substance compared to a group not treated with the candidate substance, the candidate substance may be determined to be a therapeutic substance for Alzheimer's disease.

An embodiment of the present invention may provide a kit for screening a therapeutic substance for Alzheimer's disease, including a spheroid including neurons and astrocytes, which is produced by a method including: culturing induced pluripotent stem cell-neural progenitor cells (iPSC-NPCs) derived from Alzheimer's disease patients in an NPC medium; culturing the cultured cells in a neuronal differentiation medium (NDM); and culturing the cultured cells in a glial differentiation medium (GDM).

The kit may further include any substance capable of confirming the pathological characteristics of Alzheimer's disease. For example, the kit may further include devices, reagents, and/or solvents for performing a ThT fluorescence assay to confirm Aβ (1-42) aggregates, RT-PCR analysis to confirm the degree of gene expression, or the like, but is not limited thereto.

As used herein, the “kit” refers to a product that includes various reagents required for carrying out the screening method described in the present invention and is packaged such that the reagents can be transported and stored. As suitable materials for packaging the components of the kit, glass, plastic (polyethylene, polypropylene, polycarbonate, and the like), bottles, vials, paper, bags, and the like may be used. Further, the kit according to the present invention may further include instructions for using the components of the kit according to the present invention simultaneously, sequentially or separately. The instructions may be provided in a printed form or in the form of a storage medium (for example, a magnetic disk, a memory card, an USB, and the like) as the form of an electronic document, or in a form accessible through the Internet.

“Alzheimer's disease” as described in the present invention refers to the mental impairment associated with a degenerative brain disease characterized by senile plaques, neurofibrillary tangles, and progressive neuronal loss, and is clinically manifested as progressive memory loss, confusion, behavioral problems, inability to control oneself, progressive decline in physical abilities, and eventual death. Alzheimer's disease may be divided into stages according to the Braak scale, and is characterized in that at each stage, neurofibrillary tangles accumulate starting from the transentorhinal cortex (stage I) and entorhinal cortex (stage II), and these changes progress to the limbic cortex (stage III/IV), spread to the neocortical sensory association and prefrontal areas (stage V), and eventually degenerative changes progress to the primary sensorimotor cortex.

The term “disease” refers to an abnormal condition that affects an individual's body. The disease is often construed as a medical condition associated with specific symptoms and signs. The disease may be caused by factors originally from an external source, such as infectious disease, or by internal dysfunction, such as an autoimmune disease. In humans, “disease” is often used more broadly to refer to any condition which causes pain, dysfunction, suffering, social problems, or death or similar problems in an individual suffering from the disease during contact with the individual. In a broader sense, it sometime includes injuries, disabilities, disorders, syndromes, infections, isolated symptoms, aberrant behavior, and atypical structural and functional variations, while in other contexts and for other purposes, these may be considered distinguishable categories. Diseases generally affect an individual not only physically but also emotionally, as suffering from and living with various diseases may alter the individual's perspective on life and personality.

In this context, the term “treatment” refers to the management and care of an individual for the purpose of combating a condition such as a disease or disorder, and particularly in the present invention, Alzheimer's disease. The term is intended to include the full spectrum of treatments for a given condition from which the individual is suffering, such as administration of a therapeutically effective compound to alleviate symptoms or complications, and/or to delay the progression of the disease, disorder or condition, and/or to alleviate or relieve symptoms and complications, and/or to cure or eliminate the disease, disorder or condition as well as to prevent the condition, wherein prevention is to be understood as the management and care of an individual for the purpose of combating a disease, condition, or disorder and includes the administration of an active compound to prevent the onset of symptoms or complications.

The term “therapeutic treatment” refers to any treatment that improves the health status of an individual and/or prolongs (increases) the lifespan of an individual. The treatment may eliminate the disease in the individual, stop or slow the progression of the disease in the individual, inhibit or slow the progression of the disease in the individual, reduce the frequency or severity of symptoms in the individual, and/or reduce recurrence in an individual who currently has or has had the disease.

The terms “subject” and “individual” are used interchangeably herein. These terms refer to humans or other mammals (for example, mice, rats, rabbits, dogs, cats, cows, pigs, sheep, horses, or primates), which may have or may be susceptible to a disease or disorder (for example, Alzheimer disease), but may or may not have the disease or disorder. In various embodiments, the individual is a human. Unless otherwise specified, the terms “subject and “individual” do not imply a particular age and therefore encompass adults, the elderly, children and newborns. In embodiments of the present specification, the “individual” or “subject” is a “patient.”

The term “patient” refers to a subject or individual in need of treatment, specifically a subject or individual suffering from a disease.

Reference to documents and studies referred to herein is not intended as an admission that any of the foregoing is relevant to the related art. All references to the contents of these documents are based on information available to the applicant and are not to be considered as any admission as to the accuracy of the contents of these documents.

The contents to be described below are provided to enable those skilled in the art to make and use various implementations. Descriptions of specific devices, techniques, and uses are provided by way of example only. Various modifications to the examples described herein will be readily apparent to those skilled in the art, and the general principles defined herein may be applied to other examples and applications without departing from the spirit and scope of the various embodiments. Therefore, the various embodiments are not intended to be limited to the examples described herein and shown, but are to be accorded the scope consistent with the claims.

### EXAMPLES

### Example 1. Materials and Methods

### 1.1 Culture of NPC Cells

As the iPSC-NPC cell line used in the present invention, an iPSC-NPC cell line derived from a healthy person or an Alzheimer's disease patient with a PS1 mutation (cell line ID: ax0015 or ax0112; AXOL cell line service, East Bush, UK) was used. A normal iPSC-NPC cell line and a PS1-mutated Alzheimer's disease-iPSC-NPC cell line were cultured in a dish coated with poly-L-ornithine (Sigma-Aldrich) and laminin (Sigma-Aldrich) using an NPC medium consisting of 1:100 antifungal-antibiotic (Welgene, Korea), 1:100 non-essential amino acids, sodium pyruvate (GIBCO, Waltham, MA), D-glucose (Sigma-Aldrich, Burlington, MA), L-glutamine (Welgene), 1:1000 beta-mercaptoethanol (Sigma-Aldrich), 1:50 B-27 (without vitamin A; GIBCO), and DMEM/F12 supplemented with 20 ng/ml bFGF. Accutase (STEMCELL Technologies, Vancouver, BC, Canada) was used to isolate cells.

1.2 Differentiation of iPSC-NPCs into 3D Neurons

For differentiation of iPSC-NPCs into 3D neurons, normal iPSC-NPCs or PS1-mutated Alzheimer's disease-iPSC-NPCs were seeded in a U-bottom plate (Thermo Fisher Scientific, Waltham, MA) at a density of 5×10⁴ cells and cultured in an NPC medium for 3 days. The next day, the medium was changed to a neuronal differentiation medium (NDM), and the medium was exchanged with a fresh medium once every two days for one week. The NDM includes a mixture in which DMEM F12 supplemented with 1×Glutamax (GIBCO) and a 1% B27 supplement containing no vitamin A and Neurobasal (GIBCO) are mixed at 1:1. The next day, the cell culture medium was replaced with a glial differentiation medium (GDM), and the medium was exchanged with a fresh medium once every two days for two weeks. The GDM includes a ScienCell astrocyte medium (Cat. 1801, San Diego, CA) supplemented with 1:100 antifungal-antibiotic, 1:50 bovine fetal serum, and an astrocyte growth supplement (ScienCell Cat. 1852).

1.3 Preparation and treatment of amyloid beta (Aβ) aggregates

Aggregated Aβ was prepared as described in Ryan D A, Narrow W C, Federoff H J, Bowers W J. An improved method for generating consistent soluble amyloid-beta oligomer preparations for in vitro neurotoxicity studies. J Neurosci Methods. 2010; 190(2): 171-179. More specifically, an Aβ (1-42) peptide (AGP-8338, Anygen, Gwangju, Republic of Korea) was suspended in 1,1,1,3,3,3 hexafluoro-2-propanol (HFIP; Sigma-Aldrich) at a concentration of 1 mM and stored at 20° C. until use. To form Aβ aggregates, the peptide was resuspended in DMSO at a concentration of 200 μM, sonicated in a water bath for 10 minutes, and vortexed for 30 seconds. After culturing at 37° C. for 72 hours, higher-order aggregates were prepared by culturing at 4° C. for 2 weeks. An oligomerized Aβ peptide stock was stored at −70° C. until use. When used, Aβ was suspended in PBS and used. Normal iPSC-NPCs were cultured with Aβ aggregates (10 μM) in a humidified incubator under 37° C. and 5% CO₂ conditions for 24 hours and fixed for immunocytochemistry and western blotting.

### 1.4 NDGA or CU Treatment

For drug efficacy testing of 3D cultured cells, PS1-mutated AD-iPSC-NPCs were differentiated into spheroids in a U-bottom plate (Thermo Fisher Scientific). Spheroids were cultured with nordihydroguaiaretic acid (NDGA)(10 μM, Tokyo Chemical Industry, Tokyo, Japan) or curcumin (CU)(10 μM, Tokyo Chemical Industry) in a humidified incubator under 37° C. and 5% CO² conditions for 3 days and then fixed for immunocytochemistry and western blotting.

### 1.5 ThT Fluorescence Assay

The degree of inhibition of Aβ (1-42) aggregation was measured using an in vitro ThT fluorescence assay. An Aβ (1-42) stock (0.2 mM) was prepared in 50% DMSO, and NDGA and CU stocks were prepared in 100% DMSO. Reactions for Aβ (1-42) aggregation (2 μM) and its inhibition by NDGA or CU (10 μM) were performed in a 20 mM sodium phosphate buffer (pH 8.0) supplemented with 0.2 mM EDTA and 0.02% sodium azide in the presence of 20 μM ThT in microplate wells (Corning® 96-well Black Flat Bottom Polystyrene Not Treated Microplate, Corning). ThT fluorescence intensity was measured every 30 minutes under 0.5 min shaking under 335 rpm/29.5 min incubation conditions at 37° C. using an Infinite M200 Pro (TECAN Mannedorf, Switzerland) microplate reader with 450-nm excitation and 480-nm emission filters.

### 1.6 Single-Cell Analysis for Neural Development Analysis

For single-cell analysis, a spheroid was dissociated into single cells using Accutase (STEMCELL Technologies). Single cells were analyzed through 10× genomics chromium analysis (Macrogen, Seoul, Republic of Korea). The state of neural development (immature neurons, immature astrocytes, mature neurons, or mature astrocytes) was confirmed using Loupe Browser 6.0.0.

### 1.7 Real-Time PCR for Neural Development Analysis

Each spheroid (n=3) was washed with ice-cold RNase-free PBS, and total RNA was extracted using TRIzol. Reverse transcription (RT) was conducted using Superscript reverse transcriptase (Invitrogen, Waltham, MA). Total RNA (1 μg) was used as a template for the RT reaction. Quantitative RT-PCR was performed with the SYBR™ Green system (Thermo Fisher Scientific) using a Light Cycler 480 II system (Roche, Basel, Switzerland). Amplification was monitored and measured by measuring SYBR green binding. Gene expression was calculated using the CT value, and GAPDH was used as an endogenous control.

### 1.8 Western Blot Analysis

Five spheroids were lysed in RIPA buffer (150 mM NaCl, 1% Nonidet P-40, 0.5% deoxycholic acid, 0.1% SDS, and 50 mM Tris-HCl, pH 7.4) containing a protease inhibitor (Complete Protease Inhibitor Cocktail, Roche), sonicated for 1 minute, and then cooled on ice for 20 minutes. After centrifugation at 13,200 g at 4° C. for 15 minutes, the supernatant was collected, and protein concentrations were confirmed using the BCA assay (Thermo Fisher Scientific). Equal amounts of protein were loaded on 8% to 12% SDS polyacrylamide gels. Separated samples were transferred to a PVDF membrane, cultured with primary antibodies against the target proteins, and then cultured with HRP-conjugated secondary antibodies. Protein bands were visualized by chemiluminescence (Millipore, Burlington, MA) using WesternBright™ ECL (Advansta, San Jose, CA) and detected using GBox Chemi XR5 (Syngene, Cambridge, UK). The relative intensity of each band was measured using ImageJ software (rsb. info.nih.gov).

### 1.9 Immunofluorescence

Fixed spheroids were cultured in blocking buffer (10% donkey serum and 0.3% Triton X-100 in PBS) at room temperature for 1 day. Thereafter, the samples were incubated with primary antibodies at 4° C. for 2 days and incubated with suitable fluorescent probe-conjugated secondary antibodies at room temperature for 2 days. Nuclei were stained with DAPI (1:5000; Thermo Fisher Scientific). Images were captured using a confocal microscope (FV3000, Olympus, Tokyo, Japan). The specific primary antibodies used in the present example were MAP2 (1:200, Invitrogen), GFAP (1:200, Dako), and Aβ (6E10, 1:200, BioLegend).

### 1.10 CellEvent® Caspase-3/7 Green Detection Staining

To detect cell death, spheroids were cultured in a 1 μM CellEvent® Caspase-3/7 Green detection solution (Molecular Probes, Eugene, OR) in a humidified incubator under 37° C. and 5% CO₂ conditions for 24 hours or 3 days. After being washed with a probe-free medium, samples were captured and analyzed using a fluorescence microscope (FV3000, Olympus). Cell penetration/uptake by CellEvent staining was analyzed with confocal z-stack data for the green fluorescence of activated caspase released from the inside of spheroids. The confocal z-stack data was obtained from 100 slides of each 1 μm of one spheroid.

1.11 ThT staining of Aβ aggregates in cells

To detect Aβ aggregates, spheroids were cultured in a medium containing a 10 μM ThT solution (Invitrogen) in a humidified incubator under 37° C. and 5% CO₂ conditions for 30 minutes. After washing with a probe-free medium, samples were captured and analyzed using a fluorescence microscope (FV3000, Olympus).

### Example 2. Results

### 2.1 Establishment of Conditions for 3D Culture System

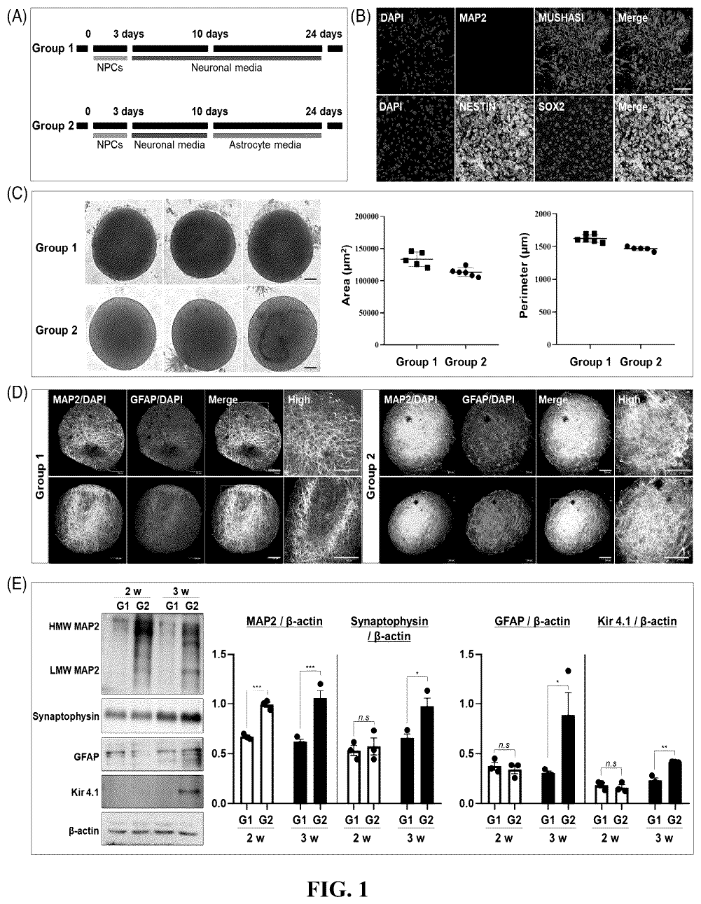

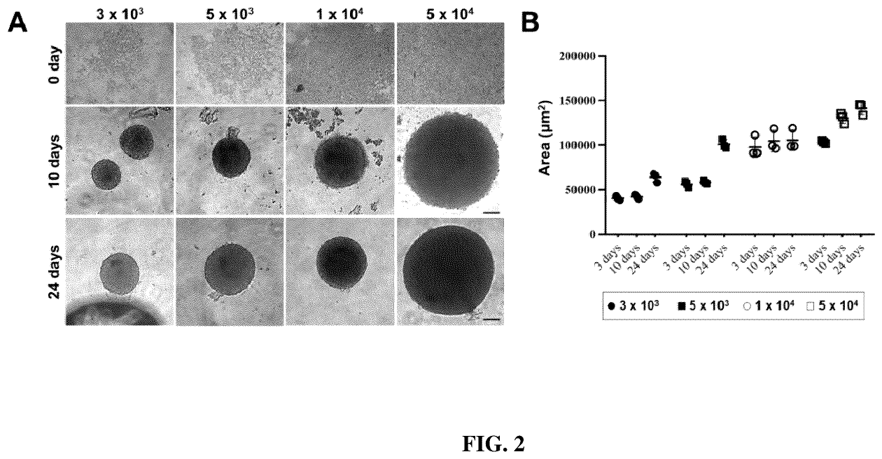

As illustrated in FIG. 1A, a 3D co-culture system of neurons and astrocytes was established using iPSC-NPCs isolated from a normal control. iPSC-NPCs isolated from normal donors were cultured under neural differentiation conditions and cultured under astrocyte generation conditions to mimic cell development and maturation processes during brain development. As normal control group iPSC-NPCs, cells that expressed MUSASHI, NESTIN, and SOX2 and did not express MAP2 were selected using immunofluorescence (FIG. 1B). As a result of comparing 3,000, 5,000, 10,000, or 50,000 cells as seed cells, the optimal number of seed cells for a stable spheroid size was confirmed to be 50,000 (FIG. 2).

To identify a suitable medium for neural differentiation, a group in which 50,000 cells were cultured in a neuronal medium alone for 3 weeks and differentiated into spheroids (Group 1) and a group in which 50,000 cells were cultured in a neuronal medium for 1 week and in an astrocyte medium for 2 weeks and differentiated into spheroids (Group 2) were compared. Although no difference in size between the two groups was found (FIG. 1C), it was confirmed that Group 2 spheroid neurons possessed more numerous dendrites expressing the mature neuron marker MAP2 and were uniformly surrounded by cells that were immunopositive for the astrocyte-specific marker GFAP (FIG. 1D). Therefore, it can be seen that Group 2 spheroids more faithfully reflected the cellular organization of the brain compared to Group 1 spheroids. Two or three weeks after differentiation in the two groups, western blots were quantified to confirm the expression of mature astrocyte markers GFAP and Kir 4.1 as well as mature neuron markers MAP2 and synaptophysin. As a result, after 3 weeks of differentiation, it was confirmed that Group 2 spheroids showed greater expression levels of MAP2, synaptophysin, and Kir 4.1 than Group 1 spheroids, as well as equivalent expression of GFAP (FIG. 1E).

In subsequent examples, Group 2 culture conditions were used to manufacture 3D neuron-astrocyte spheroids from human iPSC-NPCs.

### 2.2 Characteristics of 3D Spheroids

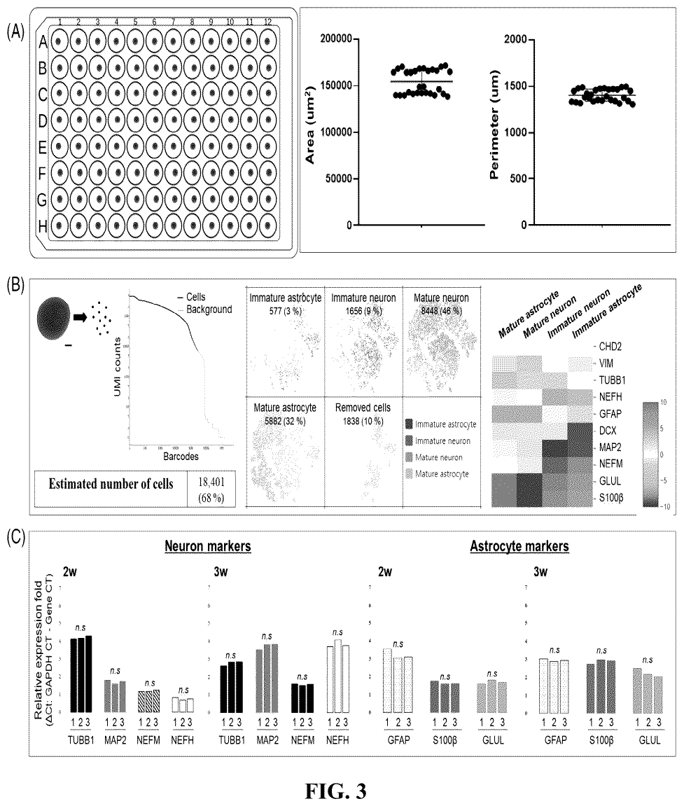

For drug efficacy testing, spheroids should be uniform in size, cellular composition, and differentiation state. In the process of 1-week neuronal cell differentiation and 2-week glial cell differentiation, the average spheroid size was very uniform (area of 154,373±2401 μm² and perimeter of 1403±12.13 μm, n=29) (FIG. 3A). The dimensions of spheroids were confirmed to be sufficient for immunostaining.

Next, the expression profile of single cells was confirmed through real-time PCR, and the uniformity of cellular composition and differentiation state was confirmed through this. Single cells (about 18,401) were isolated from each spheroid and expression levels of the immature neuron markers TUBB1 and DCX; immature astrocyte markers CHD2 and VIM; mature neuron markers MAP2, NEFM, and NEFH; and mature astrocyte markers GFAP, S100β, and GLUL were analyzed using Loupe Browser 6.0.0. Through the heatmap of gene expression, it was confirmed that a small population of cells expressed relatively high levels of CHD2 and VIM, while most of the cells expressed lower levels of CHD2 and VIM but higher levels of S100β, GLUL, and GFAP. In addition, a small population of cells expressed only TUBB1 and DCX, but most of the cells also expressed the mature neuron markers MAP2, NEFM, and NEFH (FIG. 3B). Through these results, it was confirmed that each spheroid includes about 3% immature astrocytes, about 9% immature neurons, 46% mature neurons, and 32% mature astrocytes. Furthermore, after 3 weeks of in vitro culture, RT-PCR analysis of 3 spheroids revealed no significant differences in the expression of TUBB1, MAP2, NEFM, NEFH, GFAP, S100, or GLUL, indicating substantially uniform differentiation among spheroids (FIG. 3C).

### 2.3 Detection of Cell Death and Alzheimer's Disease Pathology

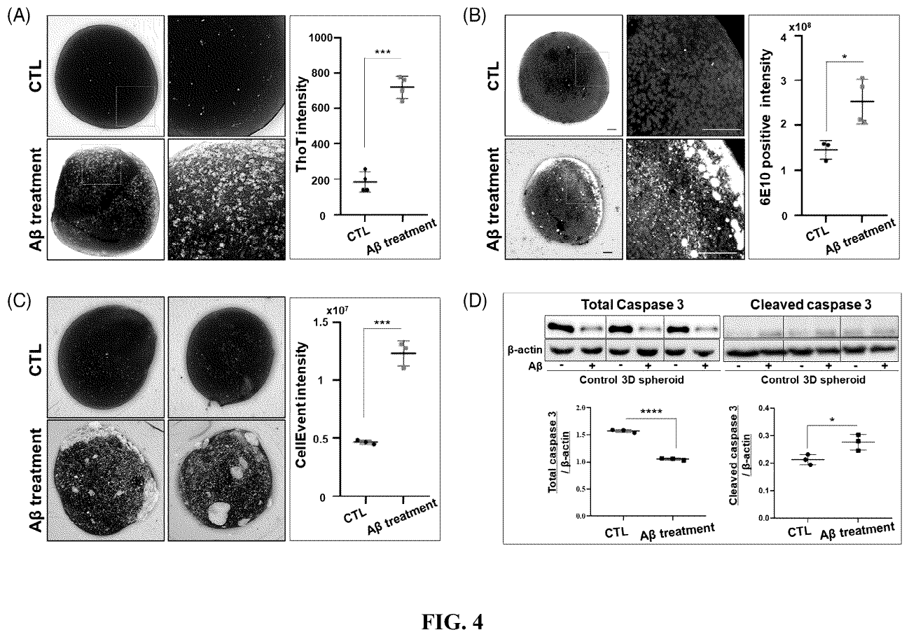

ThT staining was used to detect the accumulation of Aβ, a major therapeutic target for Alzheimer's disease, and CellEvent staining was used to detect caspase activation and cell death. When 3D spheroids manufactured from iPSC-NPCs derived from healthy individuals were treated with oligomerized Aβ, ThT fluorescence emission noticeably increased compared to 3D spheroids not treated with Aβ (FIG. 4A). The spatial distribution of ThT fluorescence did not differ substantially from that shown by using anti-Aβ immunofluorescence staining (FIG. 4B). Oligomerized Aβ treatment also substantially increased green fluorescence emission according to CellEvent staining compared to the untreated case (FIG. 4C), indicating cell death by Aβ. By western blotting, it was confirmed that the levels of total caspase 3 decreased and the level of cleaved caspase 3 increased, indicating the induction of cell death (FIG. 4D).

### 2.4 Morphology, Neural Differentiation State, and Pathological State of Alzheimer's Disease Spheroids

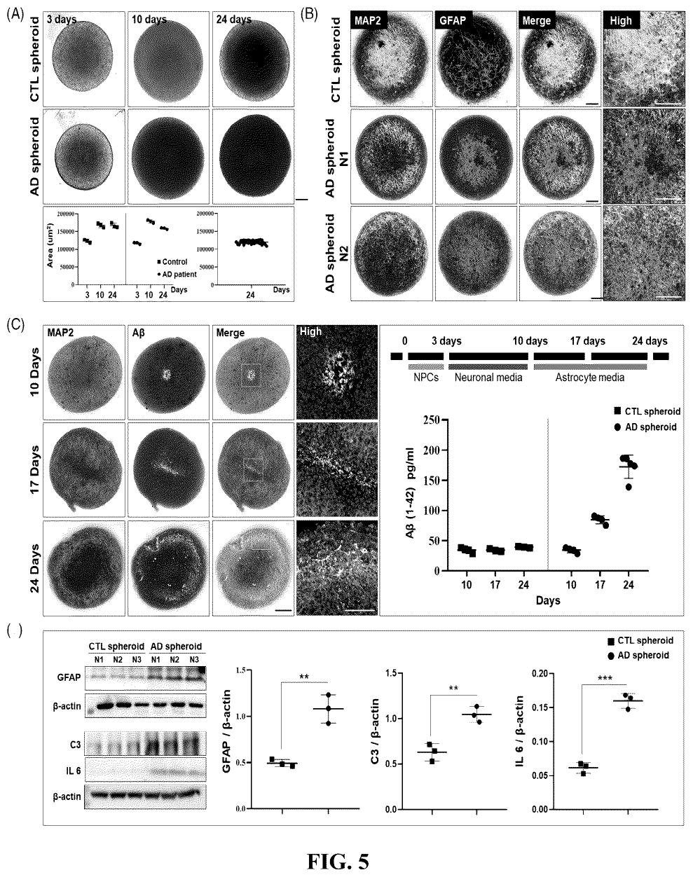

The above experiments were repeated on spheroids derived from the iPSC-NPCs of Alzheimer's disease patients. Like spheroids derived from healthy individuals, those derived from Alzheimer's disease patients also showed little differences in size and differentiation degree as indicated by immunostaining for the same phenotypic markers (FIG. 5A). However, MAP2- and GFAP-positive cells in 3D spheroids derived from Alzheimer's disease patients (cell line ID: ax0112) differed in morphological and spatial distribution compared to normal 3D spheroids (cell line ID: ax0015), and MAP-positive cells had shorter dendrites and GFAP-positive cells showed stronger GFAP expression (FIG. 5B). Through anti-Aβ immunofluorescence staining and Aβ (1-42) ELISA, it was confirmed that Alzheimer's disease-derived spheroids had gradually increased Aβ levels. Furthermore, the presence of reactive astrocytes was confirmed through enhanced expression of GFAP, C3, and IL6 on western blots. Therefore, Alzheimer's disease-derived spheroids exhibited three major pathological characteristics: reactive astrocytes; dendritic degeneration; and Aβ accumulation.

### 2.5 Reduction in Aβ Accumulation and Cell Death by Neuroprotectants in Alzheimer's Disease Spheroids

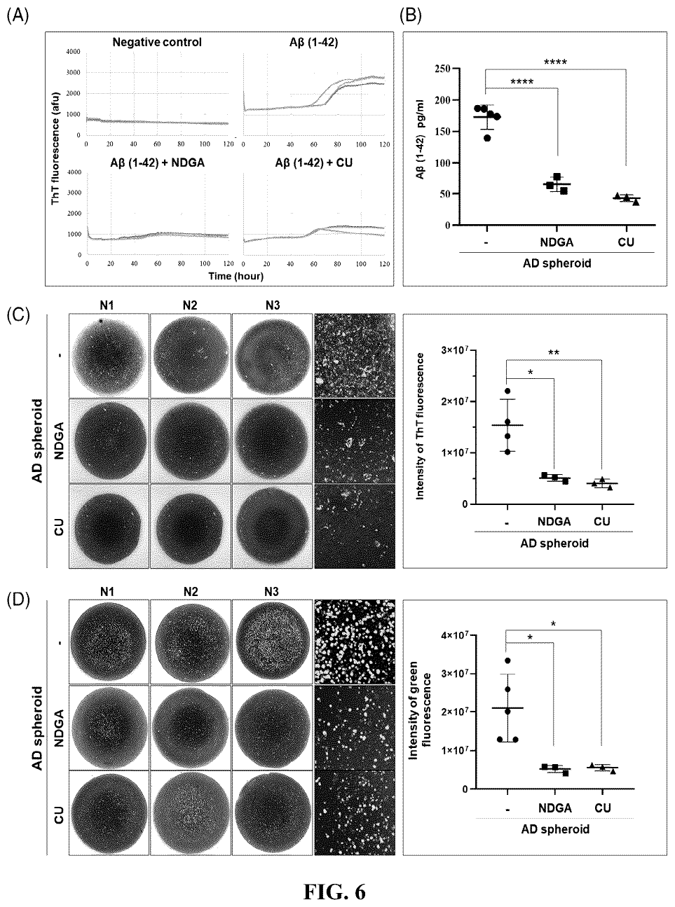

It was confirmed whether ThT fluorescence and CellEvent fluorescence in Alzheimer's disease-derived spheroids could confirm the pharmaceutical efficacy of NDGA and CU (Naiki H, Hasegawa K, Yamaguchi I, Nakamura H, Gejyo F, Nakakuki K. Apolipoprotein E and antioxidants have different mechanisms of inhibiting Alzheimer's beta-amyloid fibril formation in vitro. Biochemistry. 1998; 37(51):17882-17889; Ono K, Hasegawa K, Naiki H, Yamada M. Curcumin has potent antiamyloidogenic effects for Alzheimer's beta-amyloid fibrils in vitro. J Neurosci Res. 2004; 75(6):742-750), which are known as anti-Alzheimer compounds. In an in vitro ThT fluorescence assay for Aβ (1-42) aggregation, Aβ (1-42) alone demonstrated robust aggregation, whereas the addition of 10 μM NDGA and 10 μM CU to the reaction suppressed Aβ (1-42) aggregation (FIG. 6A). Based on the above results, Alzheimer's disease-derived spheroids were cultured with these compounds. Using ELISA, it was confirmed that NDGA and CU decreased Aβ (1-42) levels in Alzheimer's disease-derived spheroids (FIG. 6B). Further, these compounds reduced the increase in ThT and CellEvent fluorescence signals induced by oligomerized Aβ endogenously formed in Alzheimer's disease-derived spheroids, as confirmed by confocal microscopy. In addition, the changes in ThT and CellEvent fluorescence signals were qualitatively identical (FIGS. 6C and 6D). Therefore, both compounds appear to eliminate pathogenic Aβ aggregation and reduce cell death.

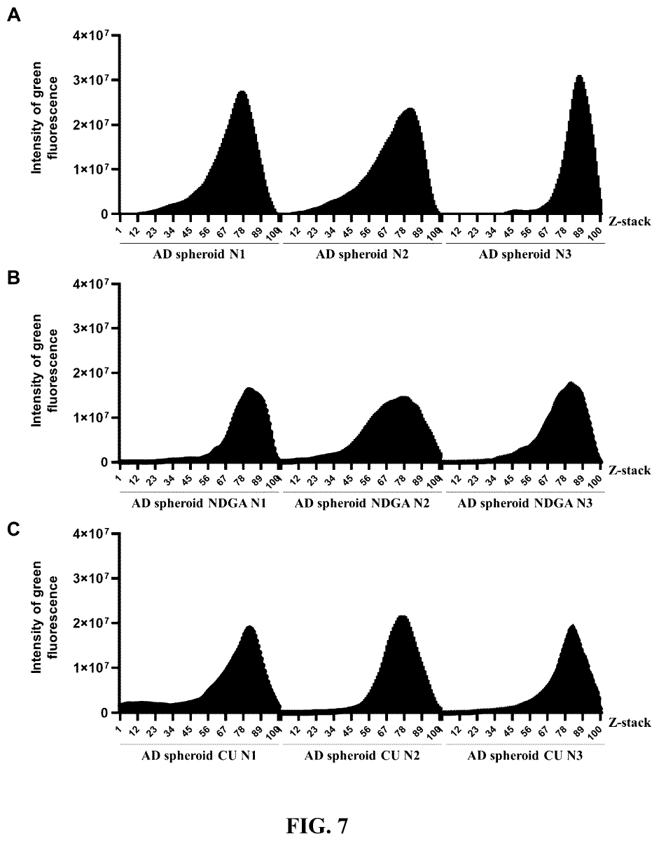

To confirm the accessibility of compounds from the surface to the center of spheroids, the fluorescence of Alzheimer's disease spheroids treated with or without NDGA or CU was confirmed. Through fluorescence intensity and confocal z-stack images, it was confirmed that the green fluorescence of activated caspase was present from the surface to the inside of spheroids in Alzheimer's disease-derived spheroids (FIG. 7). Furthermore, as an effect of NDGA or CU, a reduction in fluorescence from the surface to the inside of spheroids in Alzheimer's disease-derived spheroids occurred when treated with the corresponding compounds, indicating cell penetration/uptake of anti-Alzheimer's disease compounds and staining agents. Through this, it is shown that the 3D spheroid model derived from human iPSC-NPCs is an appropriate cell model that can easily and simply confirm the efficacy of novel drugs against Alzheimer's disease in a pharmaceutical manner.

The spheroid according to the present invention contains mature neurons and astrocytes which express a desired marker after 3 weeks of in vitro culture and has a uniform size, and thus can be used for simple visual screening to confirm drug efficacy.

For example, for claim construction purposes, the claims set forth below are not to be construed in any way narrower than their literal language, and thus example implantations from the specification should not be read as claims. Accordingly, it is to be understood that the present invention has been described by way of example and not as a limitation on the scope of the claims. Accordingly, the present invention is limited only by the following claims. All publications, issued patents, patent applications, books and journal articles, cited in the present application are each incorporated herein by reference in their entirety.

What is claimed is:

1. A method for producing a spheroid comprising neurons and astrocytes, the method comprising:
culturing induced pluripotent stem cell-neural progenitor cells (iPSC-NPCs) in a neural progenitor cell medium;
culturing the cultured cells in a neuronal differentiation medium (NDM); and
culturing the cultured cells in a glial differentiation medium (GDM).

2. The method of claim 1, wherein the iPSC-NPCs are derived from a healthy person or Alzheimer's disease patient.

3. The method of claim 2, wherein the Alzheimer's disease patient has a presenilin1 (PS1) gene mutation.

4. The method of claim 1, wherein the NPC medium includes non-essential amino acids, sodium pyruvate, glucose, glutamine, beta-methanol, and bFGF.

5. The method of claim 1, wherein the culturing in the NPC medium is performed in a U-shaped bottom plate for 2 to 4 days.

6. The method of claim 1, wherein the culturing in the NDM is performed in a U-shaped bottom plate for 6 to 8 days.

7. The method of claim 1, wherein the culturing in the GDM is performed in a U-shaped bottom plate for 12 to 16 days.

8. A spheroid comprising neurons and astrocytes, which is produced by a method comprising:
culturing induced pluripotent stem cell-neural progenitor cells (iPSC-NPCs) in an NPC medium;
culturing the cultured cells in a neuronal differentiation medium (NDM); and
culturing the cultured cells in a glial differentiation medium (GDM).

9. The spheroid of claim 8, wherein the iPSC-NPCs are derived from a healthy person or Alzheimer's disease patient.

10. The spheroid of claim 9, wherein the Alzheimer's disease patient has a presenilin1 (PS1) gene mutation.

11. The spheroid of claim 8, wherein the NPC medium includes non-essential amino acids, sodium pyruvate, glucose, glutamine, beta-methanol, and bFGF.

12. The spheroid of claim 8, wherein the culturing in the NPC medium is performed in a U-shaped bottom plate for 2 to 4 days.

13. The spheroid of claim 8, wherein the culturing in the NDM is performed in a U-shaped bottom plate for 6 to 8 days.

14. The spheroid of claim 8, wherein the culturing in the GDM is performed in a U-shaped bottom plate for 12 to 16 days.

15. The spheroid of claim 8, wherein the spheroid includes mature neurons and mature astrocytes at a ratio of 1.2:1 to 1.6:1.

16. A method for screening a therapeutic substance for Alzheimer's disease, the method comprising:
treating a spheroid including neurons and astrocytes with a candidate substance; and
confirming at least one selected from the group consisting of Aβ plaque deposition, formation of hyperphosphorylated tau, and neurodegenerative inflammation,
wherein the spheroid is produced by a method including: culturing induced pluripotent stem cell-neural progenitor cells (iPSC-NPCs) derived from an Alzheimer's disease patient in an NPC medium; culturing the cultured cells in a neuronal differentiation medium (NDM); and
culturing the cultured cells in a glial differentiation medium (GDM).

17. The method of claim 16, wherein when Aβ plaque deposition, formation of hyperphosphorylated tau, or neurodegenerative inflammation is reduced in a group treated with the candidate substance compared to a group not treated with the candidate substance, the candidate substance is determined to be a therapeutic substance for Alzheimer's disease.

18. The method of claim 16, wherein the spheroid includes mature neurons and mature astrocytes at a ratio of 1.2:1 to 1.6:1.

19. A kit for screening a therapeutic substance for Alzheimer's disease, the kit comprising: a spheroid including neurons and astrocytes, wherein the spheroid is produced by a method including:
culturing induced pluripotent stem cell-neural progenitor cells (iPSC-NPCs) derived from an Alzheimer's disease patient in an NPC medium;
culturing the cultured cells in a neuronal differentiation medium (NDM); and
culturing the cultured cells in a glial differentiation medium (GDM).

20. The kit of claim 19, wherein the spheroid includes mature neurons and mature astrocytes at a ratio of 1.2:1 to 1.6:1.
